(19) United States
(12) Patent Application Publication
(10) Pub. No.: US 2011/0178539 A1
(43) Pub. Date: Jul. 21, 2011

(54) LEFT ATRIAL APPENDAGE OCCLUSION DEVICES

(76) Inventors: David R. Holmes, Jr., Rochester, MN (US); David R. Holmes, III, Rochester, MN (US); Paul A. Friedman, Rochester, MN (US); Charles J. Bruce, Rochester, MN (US); Samuel J. Asirvatham, Rochester, MN (US); Susan B. Johnson, Rochester, MN (US); Rebecca B. Fountain, Germantown, TN (US)

(21) Appl. No.: 13/003,707

(22) PCT Filed: Jul. 8, 2009
(86) PCT No.: PCT/US09/49955

§ 371 Date: Apr. 5, 2011

Related U.S. Application Data
(62) Division of application No. 61/080,166, filed on Jul. 11, 2008.

(51) Int. Cl. A61F 2/00

(52) U.S. Cl. 606/151

(57) ABSTRACT

This document provides methods and materials related to minimally invasive techniques for reducing the volume of and/or occluding left atrial appendages.

### CROSS-REFERENCE TO RELATED APPLICATIONS

This application claims the benefit of U.S. Patent Application Ser. No. 61/080,166, filed Jul. 11, 2008. The disclosure of the prior application is considered part of (and is incorporated by reference in) the disclosure of this application.

### TECHNICAL FIELD

This document relates to materials and methods for occluding left atrial appendages.

### BACKGROUND

The left atrial appendage (LAA) is derived along with the left wall of the left atrium, which forms during the fourth week of embryonic development. The tissue making up the LAA has physiological characteristics (e.g., increased distensibility) and developmental characteristics that are distinct from the tissue in the remainder of the left atrium. The LAA is positioned in close relation to the free wall of the left ventricle. The increased distensibility and location of the LAA make it suited to function as a decompression chamber during left ventricular systole and during other periods when left atrial pressure is high. During irregular heart activity (e.g., atrial fibrillation, activity caused by mitral valve disease/damage, and the like), thrombus (blood clots) can form in the LAA. These thrombi may form due to increased stagnation of blood within the interior of the LAA. As such, removal or modification of the LAA may help to reduce the risk of thromboembolism by decreasing in size, or eliminating, the space in which blood can stagnate and later be returned into circulation.

### SUMMARY

This document provides methods and materials related to minimally invasive techniques for reducing the volume of and/or occluding the left atrial appendage. Modification of a LAA in this manner can help to reduce the risk of thromboembolism in patients with cardiac disorders.

In general, one aspect of this document features an implantable device for excluding the interior volume of a left atrial appendage of a heart from the circulation. The device comprises, or consists essentially of, an expandable housing having a first surface configured to contact the epicardial surface of the left atrial appendage, wherein the first surface of the expandable housing is configured to move a portion of the wall of the left atrial appendage toward the interior of a left atrium of the heart when the housing is in an unexpanded state, and wherein the first surface of the expandable housing is configured to expand to a size that extends past the perimeter of the ostium of the left atrial appendage at least one location of the perimeter, thereby excluding the interior volume of the left atrial appendage from communication with the left atrium. The expandable housing can comprise side walls. The side walls can be expandable. The side walls can be expandable to a lesser degree than the first surface. The first surface can be circular. The first surface can be square-shaped. The first surface can be convex. The implantable device can comprise an inflatable balloon attached to the expandable housing. The implantable device can comprise a connector attached to the expandable housing. The implantable device can comprise a clamping portion attached to the connector. The clamping portion and the connector can be configured such that the clamping portion is movable along the connector toward the expandable housing. The implantable device can lack a balloon. The implantable device can comprise a suture. The first surface of the expandable housing can be configured to expand to a size that extends past the entire perimeter of the ostium. The first surface of the expandable housing can be configured to expand to a size that extends past the perimeter of the ostium at at least one location of the perimeter, thereby securing the device to the heart.

In another aspect, this document features a method for reducing the interior volume of a left atrial appendage of a heart. The method comprises, or consists essentially of, (a) pressing an epicardial surface of the left atrial appendage toward the interior of a left atrium of the heart, thereby reducing the volume, (b) excluding the residual of the volume from the circulation, and (c) implanting a device configured to maintain at least a portion of the reduced volume.

In another aspect, this document features a method for reducing the interior volume of a left atrial appendage of a heart. The method comprises, or consists essentially of, (a) pressing an epicardial surface of the left atrial appendage toward the interior of a left atrium of the heart under conditions such that the volume is reduced and one or more portions of the left atrial appendage extends epicardially from the heart, and (b) implanting a suture around the one or more portions.

In another aspect, this document features a method for reducing the interior volume of a left atrial appendage of a heart. The method comprises, or consists essentially of, implanting a device comprising at least two opposing structures configured to clamp tissue of the left atrial appendage under conditions that reduce the volume, wherein at least one of the opposing structures is located on the endocardial surface and another of the opposing structures is located on the epicardial surface.

In another aspect, this document features a method for reducing the interior volume of a left atrial appendage of a heart. The method comprises, or consists essentially of, (a) pressing an epicardial surface of the left atrial appendage toward the interior of a left atrium of the heart to form an endocardial inversion of the left atrial appendage, thereby reducing the volume, and (b) implanting a suture around the endocardial inversion from the interior of the heart.

In another aspect, this document features an implantable device for reducing the interior volume of a left atrial appendage of a heart. The device comprises, or consists essentially of, an expandable housing having a first surface configured to contact the epicardial surface of the left atrial appendage, wherein the first surface of the expandable housing is configured to move a portion of the wall of the left atrial appendage toward the interior of a left atrium of the heart when the housing is in an unexpanded state, and wherein the first surface of the expandable housing is configured to expand to a size that extends past the perimeter of the ostium of the left atrial appendage at least one location of the perimeter, thereby reducing the interior volume of the left atrial appendage.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a cross-sectional view of an exemplary LAA.

FIG. 1B is a cross-sectional view of the LAA of FIG. 1A being deflected by an invagination device, in accordance with some embodiments.

FIG. 1C is a cross sectional view of the LAA of FIG. 1A with an expandable-plug-type LAA occlusion device deployed in the LAA, in accordance with some embodiments.

FIG. 1D is a top view of the LAA of FIG. 1A with an expandable-plug-type LAA occlusion device deployed in the LAA, in accordance with some embodiments.

FIG. 1E is a top view of a nitinol-mesh-type LAA occlusion device, in accordance with some embodiments.

FIGS. 1F-1G are top views of alternate embodiments of the LAA occlusion device of FIG. 1A employing non-circular shapes.

FIG. 1H is a cross-sectional view of an exemplary LAA.

FIG. 1I is a cross-sectional view the LAA of FIG. 1H being deflected by an inversion device, in accordance with some embodiments.

FIG. 1J is a cross sectional view of the LAA of FIG. 1H with an expandable-plug-type LAA occlusion device deployed in the LAA, in accordance with some embodiments.

FIG. 2A is a cross-sectional view of an LAA with an expandable-disc-type occlusion device deployed in the LAA, in accordance with some embodiments.

FIG. 2B is a cross-sectional view of an LAA with an umbrella-type occlusion device deployed in the LAA, in accordance with some embodiments.

FIG. 2C is a cross-sectional view of an LAA with an dual-disc-balloon-type occlusion device deployed in the LAA, in accordance with some embodiments.

FIG. 2D is a cross-sectional view of an LAA with an radially-expanding-type occlusion device deployed in the LAA, in accordance with some embodiments.

FIGS. 2E and 2F are cross-sectional views of an LAA with a double-disc-type occlusion device deployed in the LAA, in accordance with some embodiments.

FIG. 2G is a cross-sectional view of an LAA with an expanding-type occlusion device deployed in the LAA, in accordance with some embodiments.

FIG. 2H is a cross-sectional view of an LAA with a nitinol-mesh-type occlusion device deployed in the LAA, in accordance with some embodiments.

FIG. 2I is a cross-sectional view of an LAA with an patch-type occlusion device deployed against the LAA, in accordance with some embodiments.

FIG. 2J is a cross-sectional view of an LAA with an expandable-disc-type occlusion device (similar to that of FIG. 2B and including suture clips) deployed in the LAA, in accordance with some embodiments.

FIG. 2K is a cross-sectional view of an LAA with an endocardially deployed loop/suture around a portion of the LAA, in accordance with some embodiments.

FIG. 2L is a cross-sectional view of an LAA with endocardially and epicardially deployed anchors securing a portion of the LAA, in accordance with some embodiments.

FIGS. 2M, 2N, and 2O are cross-sectional views of an LAA with a nitinol-mesh-type occlusion device being deployed in the LAA, in accordance with some embodiments.

FIG. 2P is a cross-sectional view of an LAA after deflection by an invagination device, in accordance with some embodiments.

FIG. 2Q is a cross sectional view of the LAA of FIG. 2P with a coil-type LAA occlusion device deployed in the LAA, in accordance with some embodiments.

FIGS. 3A-3B are cross sectional views of a LAA with a dual-disc type LAA occlusion device is different stages of deployment in the LAA, in accordance with some embodiments.

FIG. 3C is a cross sectional view of a LAA with a dual-disc type LAA occlusion device including an additional securement mechanism, in accordance with some embodiments.

FIG. 3D is a cross sectional view of a LAA with a dual-disc type LAA occlusion device including an additional space-filling mechanism, in accordance with some embodiments.

FIGS. 3E-3F are cross sectional views of a LAA with dual-disc type LAA occlusion devices, in accordance with some embodiments.

FIGS. 4A-4C are cross sectional views of the LAA and occlusion device of FIG. 3A being deployed by a needle, in accordance with some embodiments.

Like reference symbols in the various drawings indicate like elements.

### DETAILED DESCRIPTION

For some individuals (e.g., individuals suffering from atrial fibrillation), anatomical structures within the heart, such as a LAA, can be problematic with respect to the pooling of blood, the formation of blood clots, and subsequent damage (e.g., heart attacks, strokes, and the like) that can be caused by these clots. Reduction of the size of, or occlusion/covering of a LAA can minimize the risk of clot formation and subsequent damage caused by the formed clots.

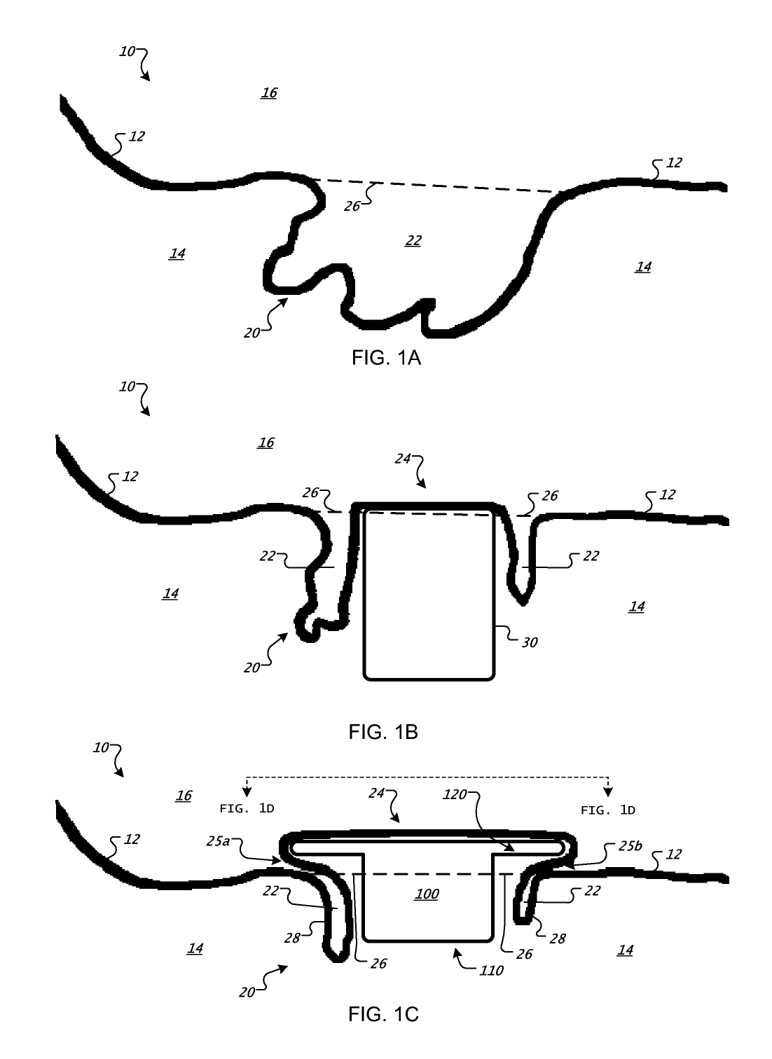

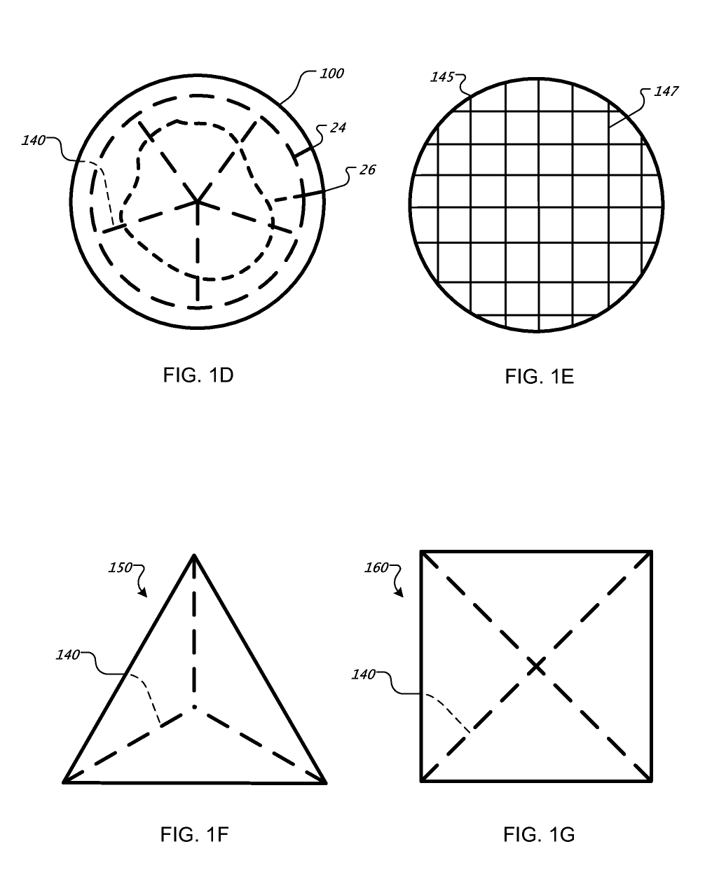

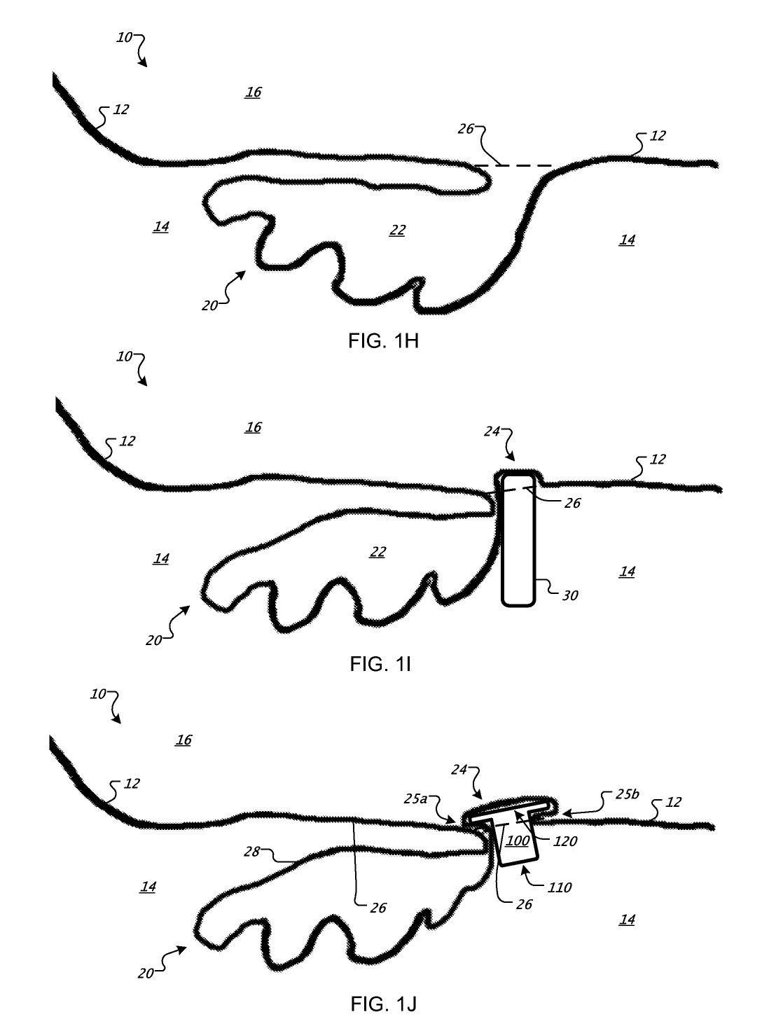

Referring now to FIG. 1A, a left atrium 10 can include a lateral wall 12 with a LAA 20 having physiological characteristics that are distinct from the other portions of the lateral wall 12 of the left atrium 10. Exemplary characteristics that distinguish the LAA 20 from the surrounding lateral wall 12 can include increased distensibility of the LAA, higher concentration of atrial natriuretic factor (ANF) granules, differing neuronal configuration, and the like. During normal heart function, the LAA 20 can expand and contract in synchronization with the left atrium 10, but to a greater degree due in part to the increased distensibility of the LAA 20. When the LAA 20 expands, an interior 22 of the LAA 20 can fill with blood, which can be emptied during subsequent contraction of the left atrium 10 and the LAA 20. During irregular heart function (e.g., atrial fibrillation, irregular function due to mitral valve disease, or the like) blood may pool and stagnate within the interior space 22, leading to the formation of blood clots. These clots can travel from the interior 22 of the LAA 20, to the interior 16 of the left atrium 10, and throughout the circulatory system, possibly resulting in heart attack or stroke. Preventing blood flow in and out of the LAA 20 by decreasing the size of, and/or occluding/covering the LAA 20 may reduce the risk of thromboembolism.

In some cases, only a small amount of the LAA can be inverted (FIGS. 1H-1J). For example, a small portion of the LAA (as seen in FIG. 1I) or a large portion of the LAA (as seen in, e.g., FIG. 1A) can be inverted depending upon, e.g., the type of device, the size of the device, and/or the desired treatment. In some cases, a device provided herein can be used to stiffen the lateral wall of the left atrium.

Referring now to FIG. 1B-1C, pressure can be applied to the LAA 20 through the use of an externally placed occlusion device 30. The inversion device 30 can approach the LAA 20 from a position external to the LAA (e.g., the epicardial/pericardial space 14) and can apply pressure to the LAA 20 causing at least a portion of the LAA 20 to prolapse toward the interior 16 of the atrium 10 into the ostium 26. The inversion device 30 can be designed in such a way as to minimize damage and avoid puncturing or piercing the LAA 20 when used. Once tissue 24 of the LAA 20 has been inverted into the ostium 26 (e.g., as shown in FIG. 1B), an occlusion device, such as a LAA occlusion plug 100, can be placed in the LAA 20. The occlusion plug 100 can retain the LAA 20 in an at least partially inverted position (e.g., as depicted in FIG. 1C), minimize or eliminate the remaining interior space 22, and/or isolate the interior space 22 of the LAA 20 from the interior space 16 of the left atrium 10. In the position depicted in FIG. 1C, blood can continue to flow within the interior 16 of the atrium 10, but may be prevented from flowing into the occluded interior space 22 of the LAA 20.

Referring now to FIG. 1C, the occlusion plug 100 can include a “mushroom” shape with a smaller proximal portion 110 and a larger distal portion 120. The occlusion plug 100 can be delivered to the ostium 26 and abut the at least partially inverted tissue 24 of the LAA 20 in an unexpanded state (not shown) that is smaller than the expanded state shown in FIG. 1C. Once delivered in the unexpanded state to the ostium 26 of the LAA 20, the occlusion plug 100 can be expanded to the state shown in FIG. 1C. In some embodiments, when the plug 100 is transitioned to the expanded state, the LAA can be further pushed inward into the interior 16 of the atrium 10, increasing the amount of the tissue 24 prolapsed into the interior 16 of the atrium 10 and decreasing one or more portions 28 of the LAA 20 remaining in the epicardial/pericardial space 14. As the occlusion plug 100 expands, the cross-sectional area of the distal portion 120 can become larger than the cross-sectional area of the ostium 26, such that portions of the inverted tissue 24 (e.g., the portions 25a and 25b) can contact the lateral wall 12 of the atrium 10. In the case of a plug 100 that has a cross-sectional area that is circular in shape (shown in FIG. 1D), a ring of tissue 24 from the LAA 20 can contact a ring shaped portion of the lateral wall 12, effectively sealing off the remaining interior space 22 of the LAA from the interior space 16 of the atrium 10. In some embodiments, the plug 100 can include cross-sectional shapes other than circular (e.g., square, rectangular, triangular, and the like) that, when expanded, can fluidly disconnect the interior space 22 from the interior space 16. With the interior space 22 fluidly disconnected from the interior space 16, blood may no longer flow from the interior space 16 to the interior space 22. If clots form within the interior space 22, these clots may not enter the interior space of the atrium 16 to be moved throughout the circulatory system, thus minimizing the risk of heart attack, stroke, and the like, caused by embolisms formed in the interior space 22 of the LAA 20.

In some embodiments, the occlusion plug 100 is a balloon-type plug, made of an expandable, biocompatible material, that can be deployed in the area of the LAA 20 in a non-expanded state. After deployment to the LAA 20, the occlusion plug 100 can be expanded by filling the interior under pressure. Exemplary materials that can be used to fill the interior of the plug 100 can include saline, silicone, expanding foam, a liquid polymer than can solidify when cured, and the like. In some embodiments, the plug 100 can include an expanding mechanism that biases the plug 100 to the expanded state shown in FIG. 1C. As explained in more detail in connection with FIG. 1D, the plug 100 can include expansion arms that bias the plug to the expanded state. Prior to deployment, the plug 100 can be stressed from the expanded state to the non-expanded state. After deployment, the force applied to transition the plug 100 to the non-expanded state can be removed, thus allowing the bias of the expansion mechanism to return the plug 100 to the expanded state.

Referring now to FIG. 1C-1D, in some embodiments, the occlusion plug 100 can have a generally cylindrical shape, with the distal end 120 having a larger diameter than the proximal end 110. When deployed, the distal end 120 of the plug 100 can invaginate a portion of the tissue 24 such that it can completely cover the ostium 26 without encroaching on blood flow within the interior 16 of atrium or from the pulmonary veins. The plug 100 can include one or more expansion arms 140 that can bias the expansion device toward the expanded state shown in FIGS. 1C-1D. In some embodiments, the expansion arms include a material that exhibits superelasticity when used in the patient's body. As such, the expansion arms can flexibly shift from a non-expanded state to an expanded state when deployed in the body. For example, the arms 140 may be formed from a length of nitinol wire or from a sheet of nitinol material, which has been processed to exhibit superelasticity below or at about a normal human body temperature, such as below or at about 37 degrees C. The nitinol material may comprise, for example, Nickel Titanium (NiTi), Niobium Titanium (NbTi), or the like. In some cases, the expansion arms 140 may include a metal material such as stainless steel, spring steel, titanium, MP35N and other cobalt alloys, or the like. In these embodiments, the expansion arms 140 can be formed from a material or materials that allow them to be reversibly adjustable from a non-deployed position to a deployed position.

Referring now to FIG. 1E, some embodiments of the occlusion device can include a woven nitinol disc 145. The woven structure could be circular (as shown in FIG. 1E), or any other shape, examples of which are shown in FIGS. 1F & 1G. The weave pattern 147 and nitinol gauge may be selected such that the device can remain flexible and deployable (through a catheter) while being rigid enough to resist forces (e.g., the pressures exerted by the left atrium) and remain in position. As with other embodiments, the nitinol disc 145 can have an atraumatic covering (fabric, polymer, etc.).

In some embodiments, the exterior surfaces of the occlusion device can include a porous, biocompatible material that can allow for tissue ingrowth. For example, the outer skin of the expandable plug can include porous polyethylene terephthalate, porous polytetrafluoroethylene, and the like. After implantation, the body can produce tissue ingrowth into the surface of the occlusion device, therefore adding additional securement to the device.

Referring now to FIGS. 1H-1J, some embodiments of the occlusion device can invert only a small amount of the LAA 20 into the interior 16 of the left atrium 10. As depicted in previous embodiments, a large amount of the tissue of the LAA 20 can be inverted and/or manipulated such that the remaining interior volume 22 of the LAA 20 can be only a small fraction (e.g., 10%, 14%, 21%, 27%, less than half, or the like) of the original volume. In other examples, such as those shown in FIGS. 1H-1J can invert only a small amount of the tissue associated with the LAA 20, such that the remaining volume 22 is greater than half of the original volume. The amount of tissue that is inverted can depend on factors such as the diameter of the ostium 26, the size of the occlusion device, the method used to secure the occlusion device in place, the size of the involution tool 30, and the like.

In use, the occlusion device can be deployed via a catheter with a lumen capable of delivering a stabilizing catheter/sheath, performing measurements (e.g., electrograms, impedance, ultrasound, pressure, and the like) and having suction capabilities to remove and potentially recirculate blood. In some embodiments, where an intercostal approach is used, it is preferable to not puncture, pierce, or in other way damage the lung, which generally lies between the chest wall and the LAA 20. Once the pleural space is entered, the lung can be mechanically displaced, for example, by using a deflectable paddle/sweeper-type catheter, inflating a balloon, injecting an inert gas such as helium to temporarily deflate the lung, wet gauze/cloth, and the like. In some cases, the pleural space need not be entered. For example, both the pleura and lung can be deflected away using the techniques described herein. In such cases, the need to leave a chest tube in place can be avoided. In some cases, the pleural space can be entered when there might be pleural or pericardial adhesions making it difficult to deflect the pleural space with the lung.

With the lung partially out of the way, direct access to the LAA can be possible. In one example, the pleural space can be entered using a dual lumen needle, through which two flexible wires can pass. One wire can be used to place an asymmetrically expanding balloon in the pleural space. The asymmetrically expanding balloon may be biased to expand to a greater degree toward the exterior and posterior of the patient. In other words, when the balloon is expanded, it can encourage the lung to move out of the pleural space, thus leaving a working space. The second wire can be used to advance, for example, a sheath, a needle, an occlusion device, and the like into the vicinity of the LAA 20. In another example, a balloon in front of and around an access sheath can be used to move the lung out of the way while the same sheath, having a lumen to be used with appropriate deflection, can be used to target the LAA and deploy a LAA occlusion device. In some embodiments, selective intubation of the right main bronchus can be used to deflate (wholly or partially) the left lung to allow placement of an access sheath. It would be apparent to one skilled in the art that there exist many methods of delivering an occluding device to a LAA, using a catheter, and not puncture or pierce the lung. In some embodiments, an access sheath can be coated with lung repellent substances (e.g. a wet sponge coating) and/or a tissue compatible/atraumatic coating.

In some embodiments, techniques for imaging for the lung, pleural space, pericardial space, LAA, LAA ostium, and the like, can be incorporated to assist in placement of the occluding device. Exemplary forms of imaging may include direct imaging (e.g., ultrasound, CT, or the like), or indirect/inferred imaging (e.g., measuring oxygen saturation, impedance, electrical signals, and the like). For example, ultrasound may be used directly to guide the catheter. This may be two-dimensional imaging and/or Doppler (e.g., as is used to check pulses) which could be implemented in a hollow tube/sheath. In examples using Doppler, an operator can identify heart sounds blood flow when in close proximity to the LAA, and/or sounds typical of pulmonary auscultation when the lungs are in the way. When respiratory interference is audible, the patient can be instructed to exhale allowing a needle that is measuring impedance and an electrocardiographic signal to be passed through the hollow Doppler sheath or guide. This can be incorporated into a timed respiratory training for the patient who will be awake (e.g., when local anesthesia is used) to control breathing and facilitate deployment. In some examples, a side arm of the sheath can have capabilities for lung deflation, lung deflection, suction, and the like, as noted above.

Referring now to FIGS. 1F-1G, embodiments of the occlusion device can include expandable plugs, such as expandable plugs 150 (FIG. 1F) and 160 (FIG. 1G) that are not generally cylindrical in shape. Expandable plug 150 can have a generally triangular shape, while plug 160 has a generally square shape. Many other shapes can be designed and utilized to cover, occlude, and/or prolapse a LAA for the purpose of preventing blood flow in and out of the LAA.

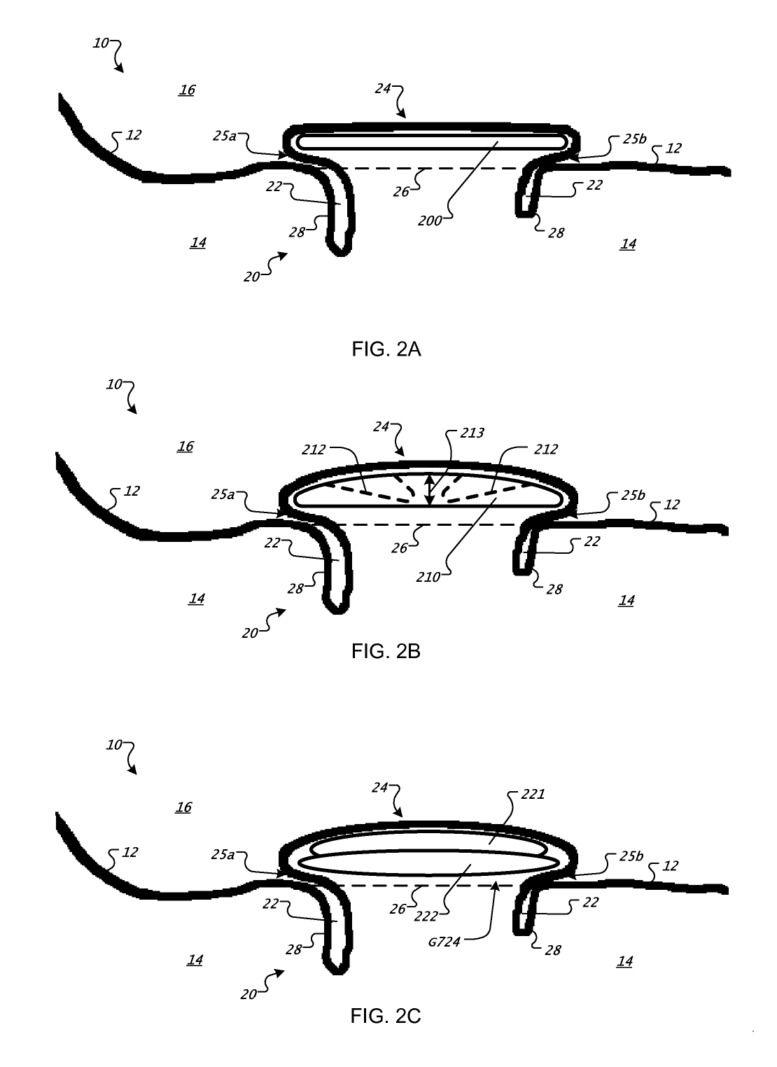

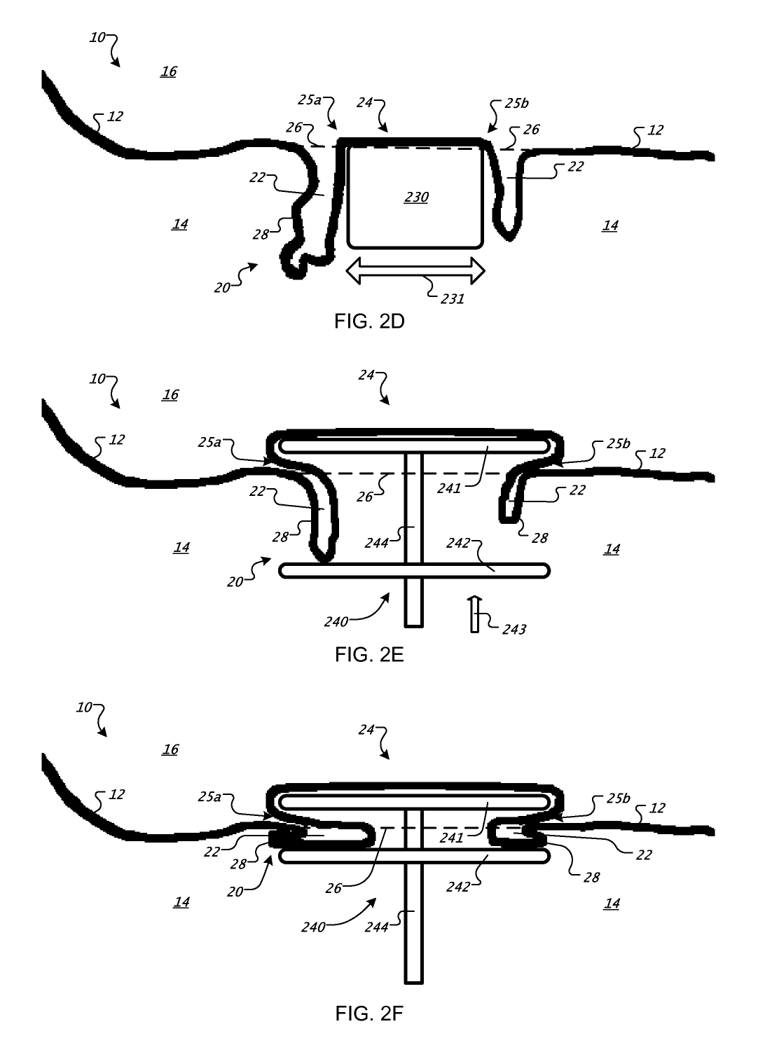

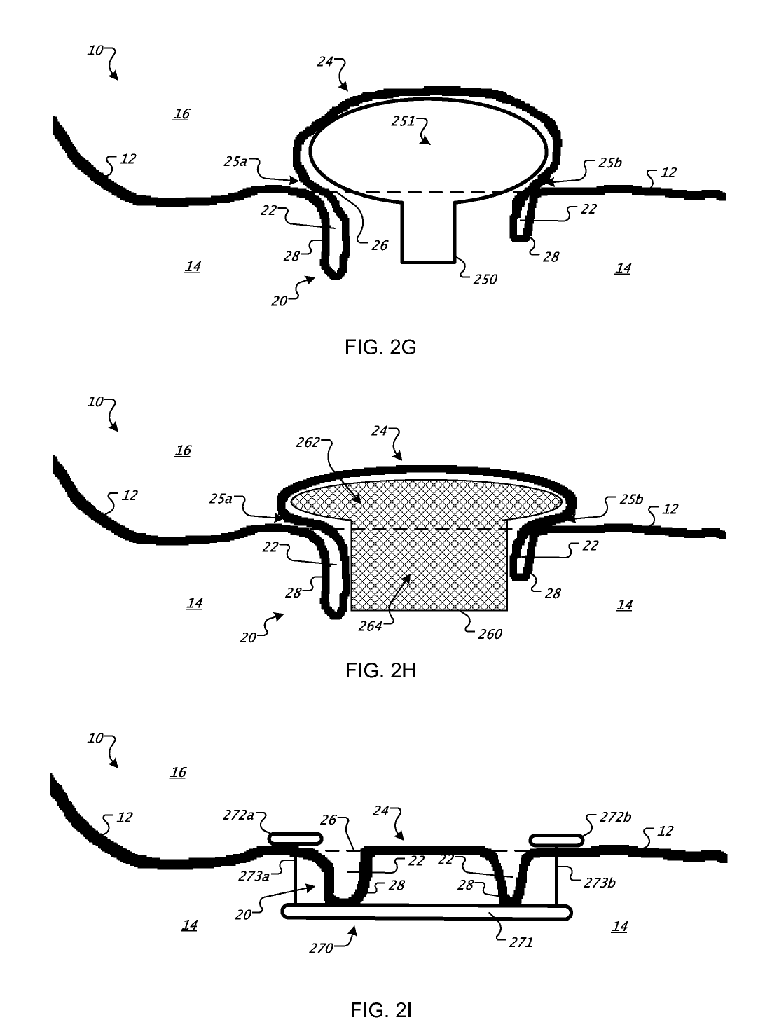

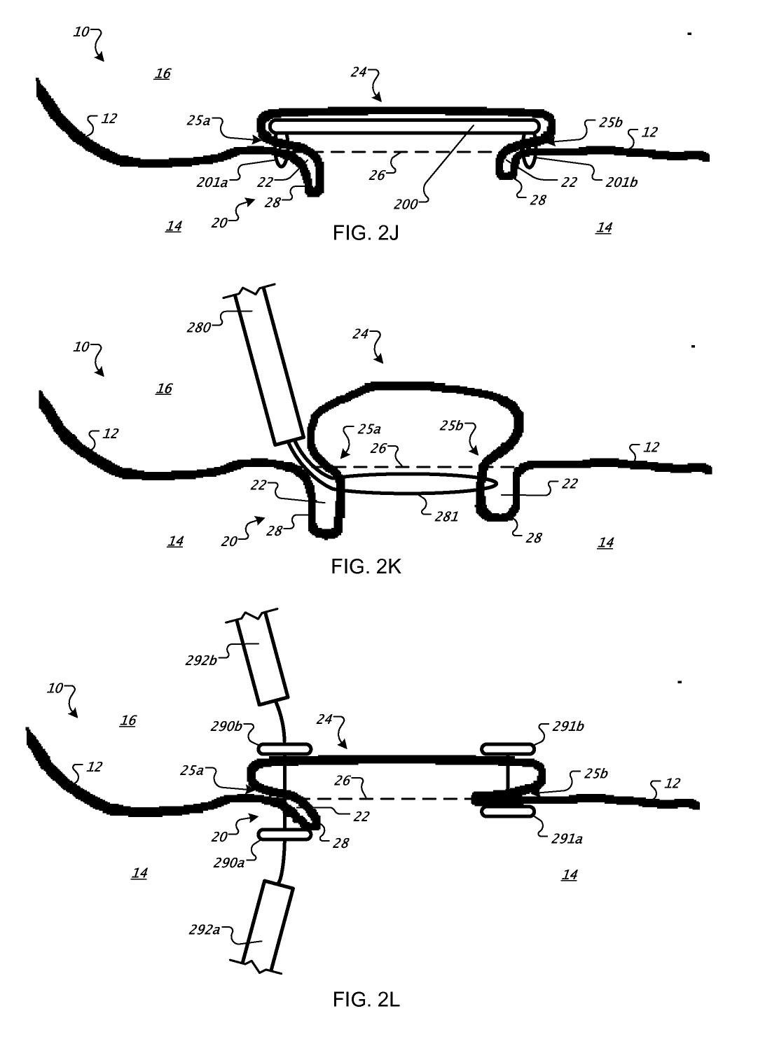

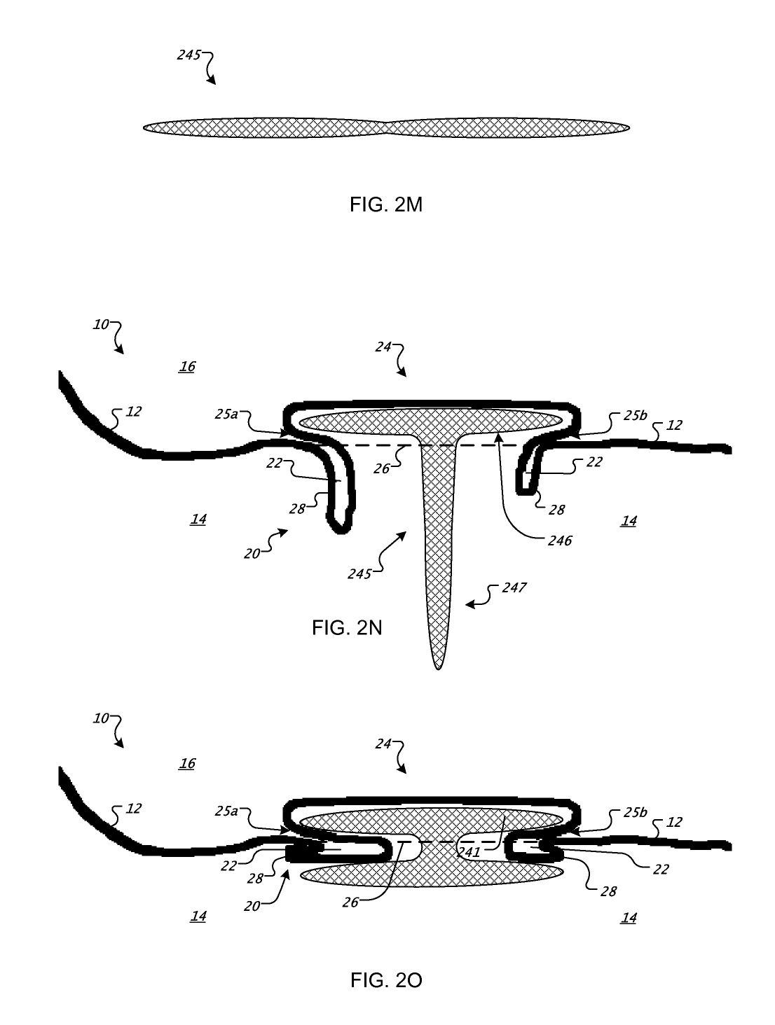

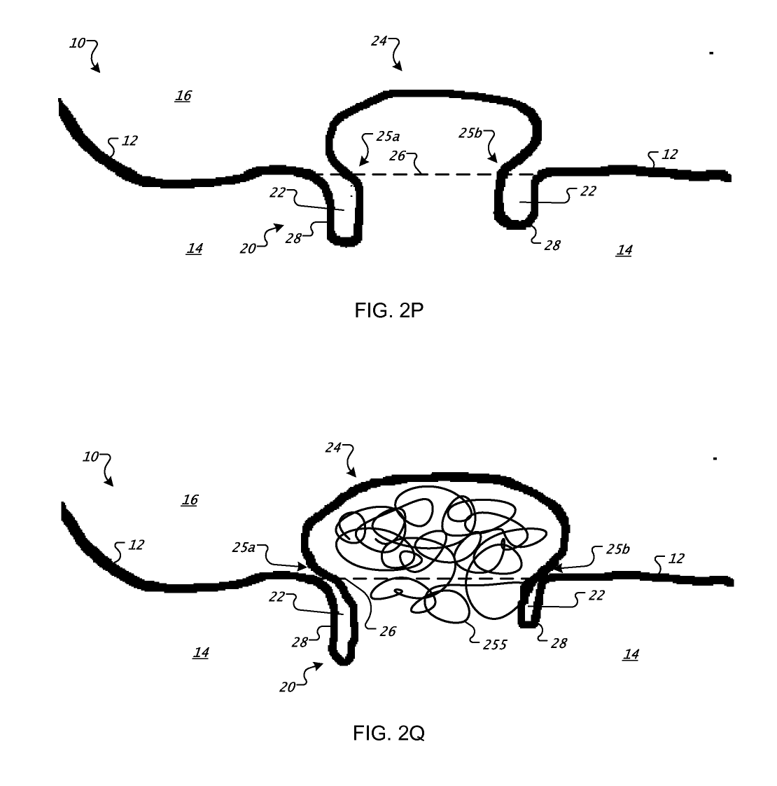

Referring now to FIGS. 2A-2L, some embodiments of the occlusion device can be used to maintain, and/or further invert, at least a portion of the LAA 20 in the interior space 16 of the atrium 10 and isolate the remaining interior space 22 of the LAA 20 from the interior space 16 of the left atrium. For example, FIG. 2A depicts an expandable disc 200 which can be delivered to the LAA 20. After use of the inversion device 30, the expandable disc 200 can be delivered to the LAA 20 in a non-expanded state (not shown), where the cross-sectional area of the expandable disc 200 in the non-expanded state is smaller than the cross-sectional area of the ostium 26. Once in place, the disc 200 can expanded (e.g., in a way that is similar to the way in which the plug 100 is expanded), to further invert a portion of the tissue 24 of the LAA 20 and cause portions of the tissue 24 (e.g., the portions 25a and 25b) to contact the lateral wall 12, thus effectively isolating the remaining interior space 22 of the LAA 20 from the interior space 16 of the left atrium.

Referring now to FIG. 2J, in some embodiments, the expandable disc 200 can be further secured to the atrium 10 through the use of securement devices such as sutures or clips (e.g., clips 201a and 202b).

Referring now to FIG. 2B, an embodiment of the occlusion device includes an umbrella device 210 that can include a mechanical device that can be used to transition the umbrella device 210 from a non-expanded state (not shown), where the cross-sectional area of the device 210 is smaller than the cross-section area of the ostium 26, to the expanded state shown in FIG. 2B, where portions of the LAA 20 can contact the lateral wall 12, thus effectively fluidly disconnecting the remaining interior space 22 of the LAA 20 from the interior space 16 of the left atrium 10. In this embodiment, the mechanical device can include arms 212 that are biased to the orientation shown in FIG. 2B. During storage and/or prior to insertion, the arms 212 can be stressed into a position that increases the longitudinal length 213 of the umbrella device 210 while decreasing the cross-sectional area of the device 210 to a size that is smaller than the cross-sectional area of the ostium 26. When deployed, the force applied to maintain the arms 212 in the stressed positions can be removed, thus allowing the bias of the arms 212 to reversibly transition the umbrella device 210 to the expanded state shown in FIG. 2B.

Referring now to FIG. 2C, an embodiment of the occlusion device can include an occlusion device 220 that includes a combination of a mechanically expandable disc 221, which is biased to a expanded state shown in FIG. 2C and a conforming/spacing filling balloon 222. For example, after use of the inversion device 30, the expandable disc 221 can be stressed to a non-expanded state (not shown), where the cross-sectional areas of the expandable disc 221 and the balloon 222 are smaller than the cross-sectional area of the ostium 26, and delivered to the LAA 20. Once in place, the disc 221 can be allowed to expand to further invert the tissue 24 of the LAA 20. After allowing the expandable disc 221 to transition to the expanded state shown, the balloon can be inflated/expanded until portions (e.g., the portions 25a and 25b) contact the lateral wall 12, thus effectively isolating the remaining interior space 22 of the LAA 20 from the interior space 16 of the left atrium. The conforming/space filling balloon can be expanded, for example, by filling it with saline, which will be retained within the balloon 222. Referring now to FIG. 2D, an embodiment of the occlusion device can include a radial expander 230 which can be retained in place through radial force applied at or within the ostium 26 of the LAA 20. For example, the radial expander 230, prior to placement in an LAA 20, can be transitioned to a non-expanded state where the radial expander 230 is smaller than the space created through the use of the inversion device 30 (not shown). Once positioned, the radial expander 230 can be expanded in the radial direction (e.g., in the directions represented by arrow 231) to the partially expanded state shown. Continued expansion of the radial expander 230 can exert force on portions of the LAA 20 (e.g., portions 25a and 25b). The expansion of the radial expander 230 can cause portions of the LAA 20 (e.g., the portions 233a and 233b) to contact the lateral wall 12 of the atrium, thus fluidly disconnecting the interior 16 of the atrium 10 from the remaining interior 22 of the LAA 20. In some embodiments, the radial expansion of the radial expander 230 can occur due to actuation of a mechanical expansion system, such as the turning of a screw, advancement of a ratchet system, and the like. The actuation of the mechanical system can cause the radius of the radial expander 230 to increase, thus displacing portions of the LAA 20. In other embodiments, the radial expander 230 may include a balloon that can be expanded by filling the balloon with, for example, saline, silicone, or the like. In still other embodiments, the expander 230 can be biased by one or more mechanical devices toward the fully expanded state (not shown). In some embodiments, the radial expander 230 can be nitinol based (e.g., constructed of a nitinol mesh) such that the expander 230 is normally biased toward the expanded state. Prior to insertion, the radial expander 230 can be stressed from the expanded state to a non-expanded state where the diameter of the expander 230 is smaller than the diameter of the ostium 26. After being positioned, the stress maintaining the device 230 in the non-expanded state can be removed, allowing the bias of the device 230 to transition it to the expanded state.

Referring now to FIG. 2E-2F, another embodiment of the occlusion device can include a double-disc system 240 delivered to the LAA 20. After use of the inversion device 30, the expandable discs 241 and 242 can be delivered to the LAA 20 in non-expanded states (not shown), where the cross-sectional areas of the expandable discs 241 and 242 are smaller than the cross-sectional area of the ostium 26. Once in place, the discs 241 and 242 can expanded. The disc 241 can further invert the LAA 20, for example, causing the inverted tissue 24 to have a diameter that is greater than that of the ostium 26. As with the embodiment described in connection with FIG. 2A, the expansion of the disc 241 can cause portions of the LAA 20 (e.g., the portions 25a and 25b) to contact the lateral wall 12 of the left atrium 20, thereby fluidly disconnecting the interior 16 of the atrium 10 from the remaining interior 22 of the LAA 20. To further secure the system 240 in place and/or increase the force sealing the LAA 20 against the lateral wall 12, the second disc 242 can be secured against the LAA 20 and/or the lateral wall 12 through the use of an adjustment mechanism 244. For example, the adjustment mechanism 244 may include teeth that can interact with a ratchet mechanism included in the second disc 242. When the discs 241 and 242 are deployed to the positions shown in FIG. 2E, force can be applied to the second disc 242 causing it to move toward the disc 241 with the direction indicated by arrow 243, while a balancing force is applied to the adjustment mechanism 244, maintaining the disc 241 against the lateral wall 12 of the left atrium 20, minimizing it's impinging of the left atrial interior space. The disc 242 can be moved until reaching the position shown in FIG. 2F. Through the combination of the adjustment mechanism 244 and the discs 241 and 242, the discs 241 and 242 can be held in the positions shown in FIG. 2F, thus securing the system 240 in place, minimizing the remaining interior space 22 of the LAA 20, and fluidly disconnecting the interior space 22 from the interior space 16 of the atrium 10. In this embodiment, the discs 241 and 242 are positioned on opposing sides of the lateral wall 12, while still remaining epicardially in that neither disc 241 nor disc 242 contact the blood. In alternate embodiments, the system 240 can be deployed from the endocardial side. In some cases, the margins at the circumference of the disc that is more external (away from the heart; e.g., disc 242) can tilt towards the disc that is relatively more internal (e.g., disc 241).

Referring now to FIGS. 2M-2O, some embodiments of the occlusion device can include a woven nitinol device 245 that can function in a similar manner to the occlusion device described in connection with FIGS. 2E-2F. In one example, the device 245 can be constructed of a nitinol mesh that is biased toward the deployed shape depicted in FIG. 2O. Prior to insertion, the device can be reversibly transitioned toward the non-deployed shape depicted in FIG. 2M, thus allowing it to be passed through, for example, a catheter lumen. Once located in the vicinity of a left atrial appendage, the catheter can be withdrawn, allowing the device 245 to begin transitioning to the deployed state. FIG. 2N depicts the device 245 where the distal portion 246 has been allowed to return to the deployed state, while the proximal portion 247 still remains in the non-deployed state (e.g., still within a catheter lumen). Further withdrawal of the catheter can allow the entire device 245 to transition to the deployed state shown in FIG. 2N.

Referring now to FIG. 2G, an embodiment of the occlusion device can include an LAA invaginated segment enlarging device 250 that can be employed to increase the size (e.g., diameter) of the inverted portion of the LAA 20 to a size (e.g., diameter) that is greater that that of the ostium 26. After use of the inversion device 30 (as described in connection with FIG. 1B), the enlarging device 250 can be delivered to the LAA 20 such that it abuts the inverted tissue 24 of the LAA 20 (not shown). Once in position, the enlarging device 250 can be expanded to increase the amount of inverted tissue 24 of the LAA 20 to the size shown in FIG. 2G. As the amount of inverted tissue 24 increases, portions 25a and 25b of the inverted tissue 24 can contact the lateral wall 12, thus fluidly disconnecting the interior 16 of the atrium 10 from the remaining interior 22 of the LAA 20. In some embodiments, the enlarging device 250 can be expanded by introducing a fluid, such as a liquid polymer, foam, or resin into the interior 251 of the enlarging device. For example, a liquid polymer can be introduced into the interior 251 to enlarge the device 250. Once the device 250 is enlarged to a point where the portions 25a and 25b contact the lateral wall 12, thus fluidly disconnecting the interior 16 of the atrium 10 from the remaining interior 22 of the LAA 20, the polymer can be allowed to cure, thus maintaining the inverted tissue 24 in substantially the position shown in FIG. 2G and effectively isolating the interior 22 of the LAA 20 from the blood located in the interior 16 of the atrium 10

In some embodiments (depicted in FIGS. 2P-2Q), metal coils (e.g., platinum coils, and the like) can be injected into the LAA 20 to maintain or increase the size (e.g., diameter) of the inverted portion of the LAA 20 to a size (e.g., diameter) that is greater that that of the ostium 26. For, the inversion device 30 can be used to invert a portion of the LAA 20 (as described in connection with FIG. 1B) to a size similar to that shown in FIG. 2P. Metal coils 255 can then be delivered to the LAA 20 such that they fill up space and maintain the LAA in the inverted position. Coils can be injected until the portions 25a and 25b contact the lateral wall 12, thus fluidly disconnecting the interior 16 of the atrium 10 from the remaining interior 22 of the LAA 20, and effectively isolating the interior 22 of the LAA 20 from the blood located in the interior 16 of the atrium 10

Referring now to FIG. 2H, an embodiment of the occlusion device can include a nitinol expanding device 260 that can be employed to secure a portion of the LAA 20 tissue in the ostium 26 and/or fluidly disconnect the interior 22 of the LAA 20 from the interior 16 of the atrium 10. After use of the inversion device 30 (as described in connection with FIG. 1B), the nitinol expanding device 260 can be delivered to the LAA 20 in an elongated, non-expanded state (similar to the elongated state depicted in FIG. 2M), where the cross-sectional area of the expanding device 260 in the non-expanded state is smaller than the cross-sectional area of the ostium 26. Once in place, the expanding device 260 can be allowed to expand (e.g., by removing a surrounding catheter), from the non-expanded state, to the normally-biased, expanded state shown in FIG. 2H. The distal portion 262 can expand to further invert a portion of the tissue 24 of the LAA 20 and cause portions of the tissue 24 (e.g., the portions 25a and 25b) to contact the lateral wall 12, thus effectively isolating the remaining interior space 22 of the LAA 20 from the interior space 16 of the left atrium, while the proximal portion 264 can expand to fill space and help maintain the device 260 in the position shown in FIG. 2H.

Referring now to FIG. 2I, an embodiment of the occlusion device can include a patch device 270 used to further collapse the LAA 20 into the interior 16 of the atrium 10, thus minimizing or eliminating the interior 22 of the LAA 20. For example, after use of the inversion device 30 (as described in connection with FIG. 1B), the patch device 270 can be applied to the LAA 20 such that a disc or patch 271 is abutted against at least a portion of the LAA 20 in the epicardial/pericardial space 14. In some embodiments, one more anchors (e.g., anchors 272a and 272b) can be secured around the perimeter of the patch 271 via securing sutures (e.g., sutures 273a and 273b). After placement of the patch 271, the anchors 272a and 272b can be inserted through the cardiac tissue of the lateral wall 12 and into the interior 16 of the atrium 10. Once inside the atrium 10, the anchors can abut the interior of the lateral wall 12 and, via the sutures 273a and 273b, hold the patch 271 in place (e.g., in the position shown in FIG. 2I. In some embodiments, the LAA 20 can be further inverted by tightening the sutures 273a and 273b, thus further minimizing or eliminating the interior 22.

Referring now to FIG. 2K, an embodiment of the occlusion device can include an endocardially deployed suture loop. For example, pressure can be applied to the LAA 20 through the use of the inversion device 30, as described in FIG. 1B. FIG. 1B depicts an embodiment where pressure is applied until at least a portion of the LAA 20 prolapses toward the interior 16 of the atrium 10 into the ostium 26. However, in the embodiment described here, pressure can be applied with the inversion device 30 until the majority of the LAA 20 prolapses into the interior 16 of the atrium 10, as shown in FIG. 2K. An endocardial catheter 280 can deploy a loop/suture 281 around the inverted tissue 24 of the LAA 20, as shown. As the loop/suture 281 is tightened, portions 25a and 25b of the LAA 20 are drawn toward each other in the directions indicated by arrows 282 until the portions 25a and 25b contact each other, thus substantially eliminating the interior 22 of the LAA 20 and securing the majority of the tissue 24 in the interior 16 of the atrium 10.

Referring now to FIG. 2L, another embodiment of the occlusion device can include a set of epicardially deployed anchors 290a and 291a and a set of endocardially deployed anchors 290b and 291b. For example, after use of the inversion device 30 (as described in connection with FIG. 1B), anchor 290a can be deployed from an epicardial catheter 292a and anchor 290b can be deployed by from an epicardial catheter 292b. When tightened, as depicted by anchors 291a and 291b, the anchors can secure a portion of the inverted tissue 24 of the LAA 20, minimize or eliminate the interior 22 of the LAA 20, and/or fluidly disconnect the interior 22 of the LAA 20 from the interior 16 of the atrium 10.

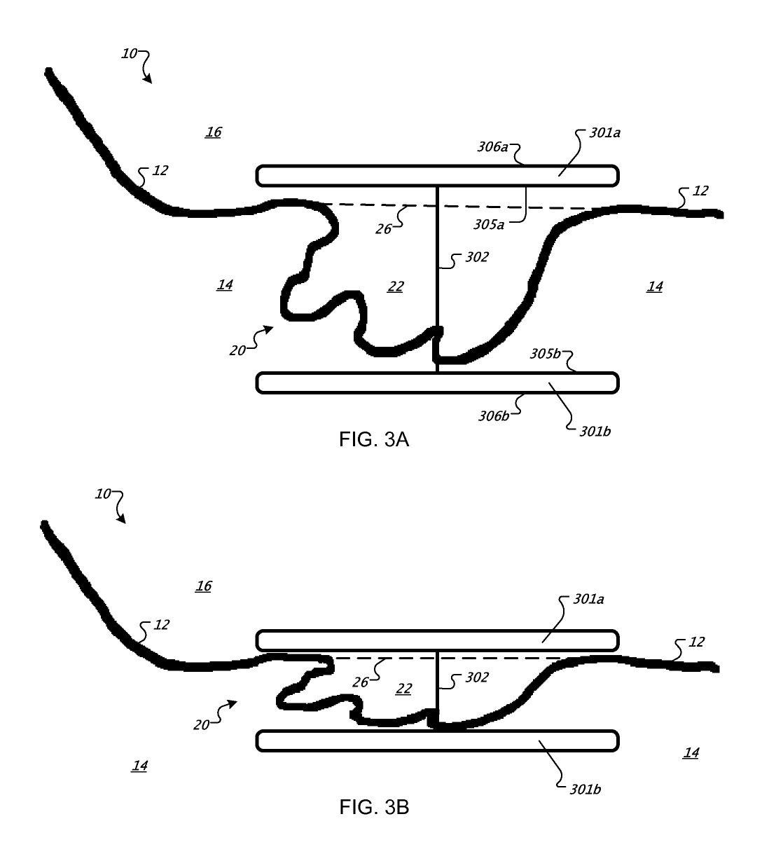

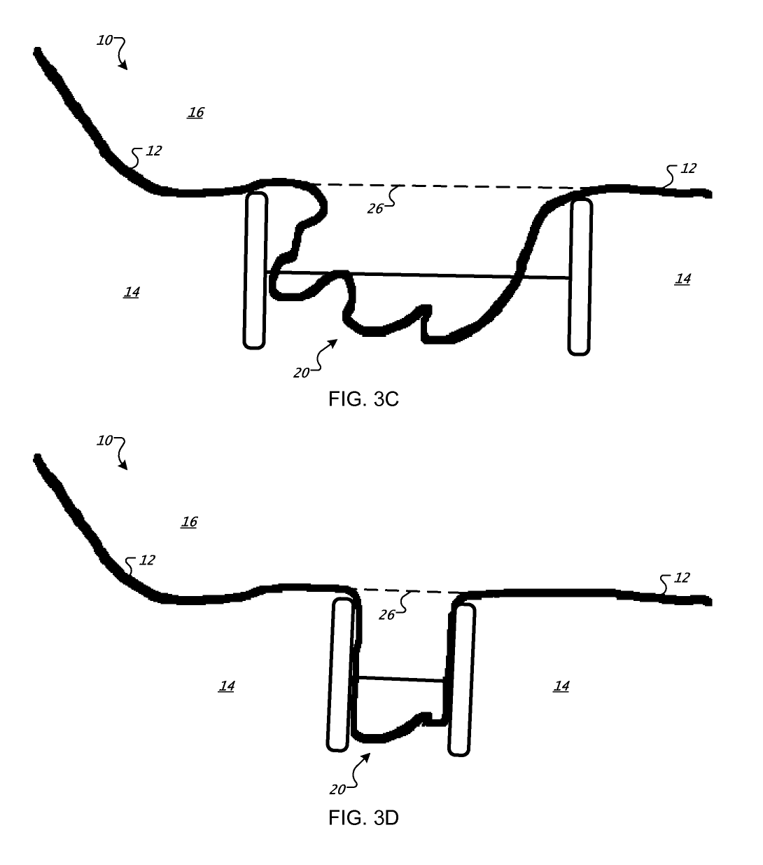

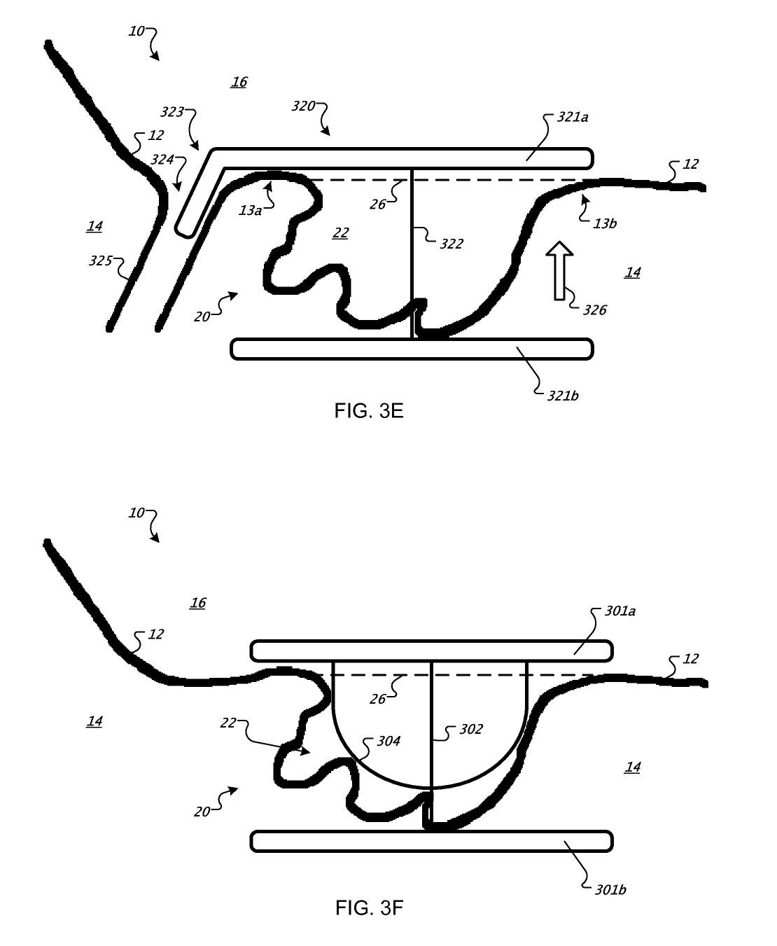

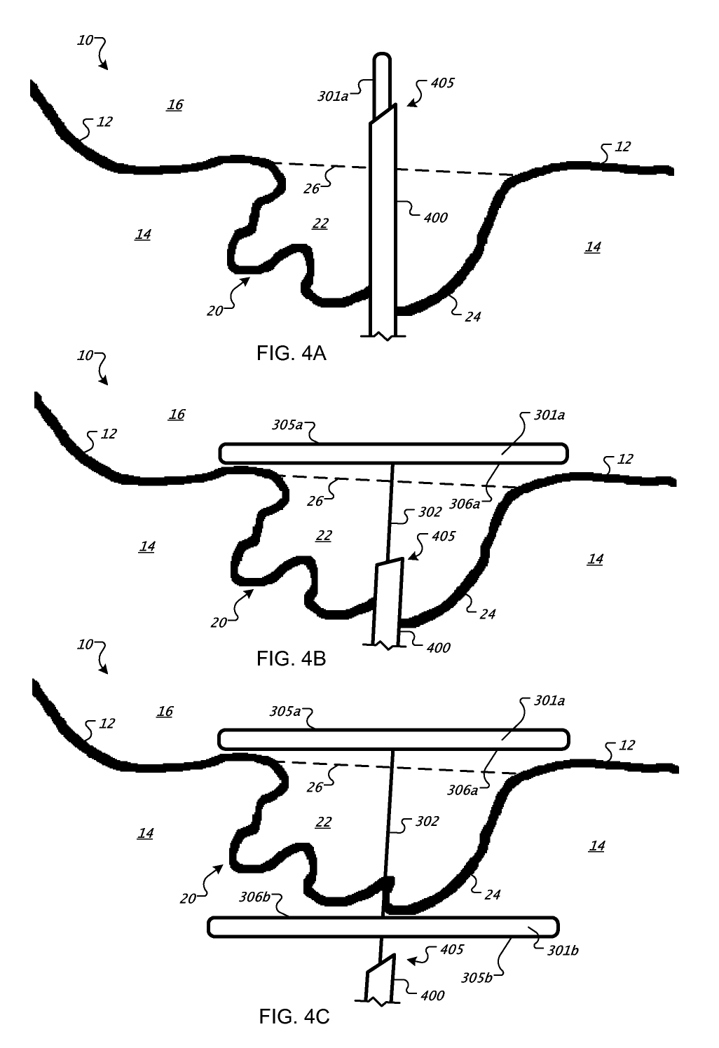

Now referring to FIG. 3A-3D, some embodiments of an occlusion device include “clam-shell” type occluding devices which can be deployed into the epicardial and/or endocardial regions (described in greater detail in connection with FIGS. 4A-4D). The occluding devices can then be used to exclude the flow of blood into the interior 22 of the LAA 20 and/or to minimize or eliminate the interior 22.

Referring now to FIG. 3A, one embodiment of a “clam-shell” occluding device 300 can include expandable discs 301a and 301b connected by adjustment member 302. For example, the expandable disc 301a can be deployed in the interior 16 of atrium 10, the expandable disc 301b can be deployed in the epicardial/pericardial space 14, with the adjustment member passing through the tissue of the LAA 20. One exemplary method of deploying the occluding device 300 will be described in more detail in connection with FIGS. 4A-4D. Once deployed as shown in FIG. 3A, discs 301a and 301b can be brought closer together using, at least in part, the adjustment member 302. As the discs 301a and 301b are brought together, at least the perimeter of disc 301a can contact the lateral wall 12 of the left atrium 10 (e.g., at portions 13a and 13b) and the disc 301b can contact the tissue of the LAA 20. Due in part to the increased distensibility of the LAA 20, as the distance between the discs 301a and 301b is decreased, the disc 301a can remain substantially stationary as the disc 301b moves toward the disc 301a (in the direction indicated by the arrow 303), thus collapsing the LAA 20. In some embodiments, the distance between the discs 301a and 301b can be decreased until reaching the positions shown in FIG. 3B. In other embodiments, the discs 301a and 301b can be brought closer together and can even be brought together until the LAA 20 is fully collapsed.

Still referring to FIG. 3A, in some embodiments, surfaces 305a and 306a of the disc 301a and surfaces 305b and 306b of the disc 301b can be substantially flat. In some embodiments, however, the surfaces 305a, 305b, 306a, and 306b can be curved, making them convex or concave. For example, the disc 301a can be curved such that the surface 305a facing the interior 22 of the LAA 20 is concave, while the surface 306a facing the interior 16 of the atrium 10 is convex. In some embodiments, the disc 302b can also be curved such that the surface 305b facing the interior 22 of the LAA 20 is concave, while the surface 306b facing the epicardial/pericardial space 14 is convex.

Referring now to FIG. 3C, one embodiment of a “clam-shell” occluding device can employ expandable discs that are both deployed in the epicardial/pericardial space and can be used to minimize or eliminate the interior of a left LAA, and/or fluidly disconnect the interior of the LAA 20 from the interior of the left atrium. For example, an occluding device 310 can include expandable discs 311a and 311b connected by adjustment member 312. The expandable discs 301a and 301b can both be deployed in the epicardial/pericardial space 14 on two sides of the LAA 20, substantially parallel to each other, but substantially perpendicular to the lateral wall 12 of the left atrium 10. In some embodiments, the adjustment member can be a pair of sutures that connect the two discs 311a and 311b and surround, but don't penetrate the LAA 20. In other examples, one or more sutures can connect the discs 311a and 311b and pass through the LAA 20. Once deployed as shown in FIG. 3C, discs 311a and 311b can be brought closer together using, at least in part, the adjustment member 312. As the discs 311a and 311b are brought together, they can remain substantially parallel to the lateral wall 12 and contacting the LAA 20. In some embodiments, the distance between the discs 311a and 311b can be decreased until reaching the positions shown in FIG. 3D. In other embodiments, the discs 311a and 311b can be brought closer together, further shrinking the interior 22, isolating the interior 22 from the interior 16 of the left atrium 10, and/or fully collapsing the LAA 20, thus eliminating the interior 22.

Referring now to FIG. 3E, one embodiment of a “clam-shell” occluding device 320 can include expandable discs 321a and 321b connected by adjustment member 322. For example, the expandable disc 321a can be deployed in the interior 16 of atrium 10 and the expandable disc 321b can be deployed in the epicardial/pericardial space 14, with the adjustment member passing through the tissue of the LAA 20. One exemplary method of deploying the occluding device 300 will be described in more detail in connection with FIGS. 4A-4D. Expandable disc 321a can include a protrusion 323 on one side that, when deployed, can be positioned in an ostium 324 of a pulmonary vein 325, such the upper pulmonary vein. When positioned, the protrusion 323 can help in anchoring the disc 321a relative to the pulmonary vein 325. Once deployed as shown in FIG. 3E, discs 321a and 321b can be brought closer together using, at least in part, the adjustment member 322. As the discs 321a and 321b are brought together, at least the perimeter of disc 321a can contact the lateral wall 12 of the left atrium 10, for example, at portions 13a and 13b as shown in FIG. 3E, isolating the interior 22 of the LAA 20 from the interior 16 of the left atrium 10. Due in part to the increased distensibility of the LAA 20, as the distance between the discs 321a and 321b is decreased, the disc 321a will remain substantially stationary, with respect to the left atrium 10, as the disc 321b moves toward the disc 321a (in the direction indicated by the arrow 326), thus collapsing the LAA 20. In some embodiments, the distance between the discs 321a and 321b can be decreased to or fluidly disconnect the interior of the LAA 20 from the interior 16 of the left atrium 10 and/or minimize or eliminate the interior 22 of the LAA 20.

Referring now to FIG. 3F, an embodiment of a “clam-shell” occluding device 300 can include a space filling device 304 that can assist in disconnecting the interior 22 of the LAA 20 from the interior 16 of the left atrium 10 and/or filling the interior 22 of the LAA 20. After deployment of the occluding device 300, the adjustment mechanism 302 can be used to compress the LAA 20 by decreasing the distance between the discs 301a and 301b. When desired, the space filling device can be expanded to seal off the interior 22 from the interior 16 of the atrium 10 and/or minimize or eliminate the interior 22. In some embodiments, the space filling device 304 can be biased to the expanded state depicted in FIG. 3F. In these embodiments, prior to expanding the space filling device 304, the space filling device 304 can be stressed into a non-expanded state for delivery. When desired, the stress maintaining the space filling device 304 in the non-expanded state can be removed, thus causing the device 304 to return to the expanded state shown. In some embodiments, the space filling device can be a structure (e.g., a balloon) that can normally be in a non-expanded state (not shown). When desired, the space filling device 304 can be filled (e.g., with saline, silicone, or the like) causing it to expand to the state shown in FIG. 3F.

In some cases, the margins at the circumference of one or more discs for a “clam-shell” device provided herein can be configured to tilt towards the other disc. For example, both discs of a “clam-shell” device provided herein can be configured such that a portion at the circumference of each disc can tilt toward a portion of the other disc.

In one exemplary use, depicted in FIGS. 4A-4C, a fine needle 400 can be used to deploy an LAA occlusion device, such as the “clam-shell” occluding device 300 around the LAA 20. In this example, the heart can be accessed from an epicardial position using the needle 400, which can be advanced from the intercostal space (e.g., third, fourth, or fifth between the mid-clavicular and posterior axillary lines) through the tissue 24 of the LAA 20 and into the interior 16 of the left atrium 10. Referring to FIG. 4A, when the tip 405 of the catheter 400 is located in the left atrium 10, the expandable disc 301a can be deployed from the tip 405 until fully deployed as shown in FIG. 4B. As the needle 400 is withdrawn from the interior 16 of the atrium 10 into the interior 22 of the LAA 20 (e.g., as depicted in FIG. 4B), the adjustment mechanism 302 can be deployed from the needle 400. The disc 301a can be pulled back until flow into the interior 22 of the LAA 20 is excluded. As the needle 400 is withdrawn, the adjustment mechanism 302 will continue to deploy from the tip 405. Referring now to FIG. 4C, at a point after the needle 400 is withdrawn from the LAA 20 into the epicardial/pericardial space 14, the expandable disc 301b of the occlusion device 300 can begin to be deployed from the needle 400 into the epicardial/pericardial space 14. The two disc 301a and 302b of the occlusion device 200 can be brought closer together with a ratchet, screw, or sliding mechanism to completely exclude flow into the interior 22 LAA 20 and/or to collapse the LAA 20 until the interior 22 is minimized or eliminated.

In some cases, the expandable devices provided herein can contain expandable portions that are not only radially expandable. For example, the entire device can go from being a cylinder to a cone shape with the larger diameter portion of the cone shape being internal to the ostium (but either internal or external to the atrium itself) and the point or smaller diameter portion of the cone shape being external to the ostium. Such devices can be deployed in a manner such that when the device is ratcheted or effectuated using a mechanism to expand the internal portion, the external portion can become smaller. In some cases, an unexpanded device can resemble a cylinder that, when effectuated, the device expands internally but externally as well either radially or in a fairly gradual expansion so it resembles, for example, a dumbbell.

While the previous embodiments describe the application of external pressure to invert and/or obliterate a left atrial appendage, followed by securing of the appendage, similar techniques can be applied to other appendage like structures to prevent fluid communication of an interior of a structure with a main lumen or visceral cavity. Exemplary applications can include the gallbladder, appendage, diverticula, pseudoaneurysms of the ventricle, pharyngal pouches and peripheral veins, diverticulae or aneurysmally enlarged veins/varices, and the like.

It is noted that a LAA occlusion device can include any of the features, improvements, and alterations disclosed herein, in any combination.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An implantable device for excluding the interior volume of a left atrial appendage of a heart from the circulation, wherein said device comprising an expandable housing having a first surface configured to contact the epicardial surface of said left atrial appendage, wherein said first surface of said expandable housing is configured to move a portion of the wall of said left atrial appendage toward the interior of a left atrium of said heart when said housing is in an unexpanded state, and wherein said first surface of said expandable housing is configured to expand to a size that extends past the perimeter of the ostium of said left atrial appendage at least one location of said perimeter, thereby excluding said interior volume of said left atrial appendage from communication with the left atrium.

2. The device of claim 1, wherein said expandable housing comprises side walls.

3. The device of claim 2, wherein said side walls are expandable.

4. The device of claim 2, wherein said side walls are expandable to a lesser degree than said first surface.

5. The device of claim 1, wherein said first surface is circular.

6. The device of claim 1, wherein said first surface is square-shaped.

7. The device of claim 1, wherein said first surface is convex.

8. The device of claim 1, wherein said implantable device comprises an inflatable balloon attached to said expandable housing.

9. The device of claim 1, wherein said implantable device comprises a connector attached to said expandable housing.

10. The device of claim 9, wherein said implantable device comprises a clamping portion attached to said connector.

11. The device of claim 10, wherein said clamping portion and said connector are configured such that said clamping portion is movable along said connector toward said expandable housing.

12. The device of claim 1, wherein said implantable device lacks a balloon.

13. The device of claim 1, wherein said implantable device comprises a suture.

14. The device of claim 1, wherein said first surface of said expandable housing is configured to expand to a size that extends past the entire perimeter of said ostium.

15. The device of claim 1, wherein said first surface of said expandable housing is configured to expand to a size that extends past the perimeter of said ostium at least one location of said perimeter, thereby securing said device to said heart.

16. A method for reducing the interior volume of a left atrial appendage of a heart, wherein said method comprises:
(a) pressing an epicardial surface of said left atrial appendage toward the interior of a left atrium of said heart, thereby reducing said volume,
(b) excluding the residual of said volume from the circulation, and
(c) implanting a device configured to maintain at least a portion of said reduced volume.

17. A method for reducing the interior volume of a left atrial appendage of a heart, wherein said method comprises:
(a) pressing an epicardial surface of said left atrial appendage toward the interior of a left atrium of said heart under conditions such that said volume is reduced and one or more portions of said left atrial appendage extends epicardially from said heart, and
(b) implanting a suture around said one or more portions.

18. A method for reducing the interior volume of a left atrial appendage of a heart, wherein said method comprises implanting a device comprising at least two opposing structures configured to clamp tissue of said left atrial appendage under conditions that reduce said volume, wherein at least one of said opposing structures is located on the endocardial surface and another of said opposing structures is located on the epicardial surface.

19. A method for reducing the interior volume of a left atrial appendage of a heart, wherein said method comprises:
(a) pressing an epicardial surface of said left atrial appendage toward the interior of a left atrium of said heart to form an endocardial inversion of said left atrial appendage, thereby reducing said volume, and
(b) implanting a suture around said endocardial inversion from the interior of said heart.

20. An implantable device for reducing the interior volume of a left atrial appendage of a heart, wherein said device comprising an expandable housing having a first surface configured to contact the epicardial surface of said left atrial appendage, wherein said first surface of said expandable housing is configured to move a portion of the wall of said left atrial appendage toward the interior of a left atrium of said heart when said housing is in an unexpanded state, and wherein said first surface of said expandable housing is configured to expand to a size that extends past the perimeter of the ostium of said left atrial appendage at least one location of said perimeter, thereby reducing said interior volume of said left atrial appendage.
